(19) United States
(12) Patent Application Publication
(10) Pub. No.: US 2015/0306165 A1
(43) Pub. Date: Oct. 29, 2015

(54) PLANT EXTRACT COMPOSITION FOR INHIBITING ADIPOCYTES, REDUCING BODY FAT, PROMOTING WEIGHT LOSS AND INCREASING LIPID METABOLISM AS WELL AS APPLICATION THEREOF

(71) Applicant: CALIWAY BIOMEDICAL CO., LTD., Taipei City (TW)

(72) Inventor: Yu-Fang LING, Taipei City (TW)

(21) Appl. No.: 14/690,915

(22) Filed: Apr. 20, 2015

Related U.S. Application Data
(60) Provisional application No. 61/984,138, filed on Apr. 25, 2014.

(51) Int. Cl. A61K 36/82; A61K 36/42; A61K 36/605; A61K 31/05; A61K 36/74

(52) U.S. Cl. CPC A61K 36/82; A61K 31/05; A61K 36/74; A61K 36/605; A61K 36/42

(57) ABSTRACT

The present invention provides a plant extract composition for inhibiting adipocytes, reducing body fat, losing body weight or increasing lipid metabolism, wherein the composition comprises 1.5 wt % to 40 wt % of resveratrol and 10 wt % to 85 wt % green tea extract. The composition not only provides high safety, but also effectively suppresses preadipocytes, inhibits differentiating adipocytes, inhibits mature adipocytes, increases the activity of carnitine acyl transferase-1, decreases body fat and promotes weight loss, and regulates the expression of the leptin gene and resistin gene.

### CROSS REFERENCE

This application claims priority to U.S. Provisional Patent Application Ser. No. 61/984,138, filed on Apr. 25, 2014, the content of which is hereby incorporated by reference in its entirety.

### BACKGROUND OF THE INVENTION

1. Field of the Invention

The present invention relates to a plant extract composition, and more particularly to the plant extract composition for inhibiting adipocytes, reducing body fat, promoting weight loss or increasing lipid metabolism. The present invention also relates to an application for inhibiting adipocytes, reducing body fat, promoting weight loss or increasing lipid metabolism. The present invention also relates to a pharmaceutical composition, and more particularly to the pharmaceutical composition comprising the above-said plant extract composition.

2. Description of the Prior Arts

According to the World Health Organization (WHO) report, obesity is a global pandemic disease wherein more than 1.4 billion adults are overweight (BMI>25), and at least five hundred million of them are obese (BMI>30). These obese people suffered from high blood pressure, dyslipidemia, diabetes, gout, and metabolic diseases at sharply higher probabilities than the average.

According to the report of the Johns Hopkins University Bloomberg School of Public Health published in 2007, approximately 75% and 41% adults would be overweight and obese, respectively, in USA in 2015. In addition, WHO provided the population of overweight and obese adults was increased from 25% to 38.5% in China in last decade. Moreover, in 2015, the overweight population will be 50% to 57% in China.

The causes of obesity are complex which multiple factors such as genetics, metabolic disorders, biochemistry, and cultural and spiritual community are involved. More and more evidences show that obesity is not only a simple self-control problem, but also involving multiple appetite regulation and energy metabolism. In addition, studies have shown that obesity is a crucial risk factor to hypertension, dyslipidemia, diabetes, cardiovascular disease, cancer, fatty liver, metabolic syndrome, and other diseases. Obesity not only increases mortality and huge burden to mankind, but also affects life quality. In general, obesity has been considered a lipid accumulation disease that is harmful for health.

Accordingly, many causes of death are considered to be correlated with obesity including cancer, cardiovascular disease, diabetes, chronic lower respiratory diseases, chronic hepatic disease and cirrhosis, renal disease, and etc makes it a global issue. Recently, the prevalence of obesity is rising accompanied by the metabolic abnormality in blood pressure, blood sugar, insulin resistance, and dyslipidemia which gradually leads to the incidence of diabetes, renal disease, atherosclerosis, cerebral thrombosis, stoke, myocardiac infarction, and eventually to death. The research of WHO also indicated that obesity is the top 5 risk factor of cause of death in the world while more than 2.8 million adults were killed by overweight or obesity per year. 44% and 23% medical burden among diabetes and ischemic heart disease respectively was attributed to obesity.

There are many weight loss products on the market while the composition, price, and quality are diverse. Currently, legitimate ingredient Orlistat® for weight loss is used for blocking intestinal absorption of partial fat to reduce a part of calories from the food. However, Orlistat® cannot reduce lipid accumulation or promote fat metabolism, so it is difficult to resolve the fundamental problem of obesity or issues derived from obesity. Moreover, Orlistat® has significant side effects, including the interference of fat-soluble vitamin absorption, oil stool, bloating, and gallstones, and may also cause liver and kidney damage, etc. Therefore Orlistat® is not suitable for long-term use. In view that the obesity problem in the United Sates is severe, two weight-loss drugs, respectively phentermine-topiramate compound and Lorcaserin, were approved in 2012 in the United States. The mechanisms of said two drugs are mainly increasing the satiety to suppress the appetite for the purpose of weight loss. Compared with Orlistat®, although said two drugs have no side effects such as gastrointestinal discomfort and oily stool, there is still dizziness, nausea, insomnia, or constipation symptom; the effect of long-term weight loss is also not obvious. Phentermine has led to serious cardiovascular disease in the past such that it is banned in many countries. The two weight-loss drugs are not suitable for patients with cardiovascular disease, and the risk of them still need longer time of monitoring. Therefore, the industry is still in dire need of a drug that has high security and low side effects and can reduce weight and regulate body fat metabolism (including reduction of visceral fat and subcutaneous fat) to solve the fundamental problem of obesity.

There are literatures indicating that green tea, mulberry leaf, resveratrol, or bitter gourd and extracts thereof have efficacy such as antioxidant, anti-inflammatory, lowering cholesterol, lowering blood sugar, and preventing cardiovascular and cerebrovascular diseases. However, it is not revealed that above-said plants or extracts thereof can increase the activity of carnitine acyl transferase-1 (CPT-1). By means of CPT-1, long-chain fatty acids can combine with carnitine to go into mitochondria for fat metabolism and energy production. When the activity of the CPT-1 is increasing, it can promote body fat consumption and reduce body fat, so mitochondria are considered as a plant for metabolism in cell.

Resveratrol is a polyphenolic compound found mainly in the skin of red grapes, Polygonum cuspidatum or red wine. Currently research indicates that resveratrol can help resist free radical damage, reduce inflammation, and improve cardiovascular disease, diabetes and metabolic syndrome, and other related diseases.

Although there have been described in the adipocytes experiments that resveratrol can effectively inhibit lipid accumulation of mature fat cells or promote the growth of preadipocytes, feeding mice with resveratrol cannot significantly promote weight loss or body fat loss (including visceral fat and subcutaneous fat) in experiments that the mice are fed with high fat diet and resveratrol continuously.

### SUMMARY OF THE INVENTION

To overcome the shortcomings as mentioned above, the objective of the present invention is to provide a plant extract composition and application thereof. The plant extract composition in accordance with the present invention comprises 1.5 wt % to 40 wt % resveratrol and 10 wt % to 85 wt % green tea extract. Preferably, the plant extract composition further comprises an extract selected from the group consisting of green coffee bean extract, mulberry leaf extract and bitter gourd extract. The plant extract composition of the present invention can effectively inhibit preadipocytes, differentiating adipocytes and mature adipocytes, increase the activity of CPT-1, and regulate the expression levels of the adipokines such as leptin and resistin to reduce body fat and to promote weight loss.

The present invention provides a plant extract composition for inhibiting adipocytes, reducing body fat, promoting weight loss and increasing lipid metabolism, wherein the plant extract composition comprises 1.5 wt % to 40 wt % resveratrol and 10 wt % to 85 wt % green tea extract.

Preferably, the plant extract composition comprises 10 wt % to 30 wt % resveratrol.

Preferably, the plant extract composition comprises 40 wt % to 80 wt % green tea extract.

Preferably, the plant extract composition further comprises an extract selected from the group consisting of green coffee bean extract, mulberry leaf extract and bitter gourd extract, wherein green coffee bean extract is from 0 wt % to 40 wt %, mulberry leaf extract is from 0 wt % to 40 wt %, and bitter gourd extract is from 0 wt % to 65 wt %.

Preferably, the plant extract composition comprises 20 wt % to 40 wt % green coffee bean extract.

Preferably, the plant extract composition comprises 10 wt % to 40 wt % mulberry leaf extract.

Preferably, the plant extract composition comprises 17 wt % to 50 wt % bitter gourd extract.

According to the present invention, the term “inhibiting adipocytes” as used herein refers to inhibition of preadipocytes, differentiating adipocytes, and mature adipocytes growth. According to the present invention, inhibiting preadipocytes is determined by administering various concentrations of the plant extract composition to preadipocytes in a specific period of time. According to the present invention, inhibiting differentiating adipocytes is determined by inducing preadipocytes differentiation and then administering various concentrations of the plant extract composition to differentiating adipocytes in a specific period of time. According to the present invention, inhibiting mature adipocytes is determined by inducing preadipocytes into mature adipocytes and then administering various concentrations of the plant extract composition to mature adipocytes in a specific period of time.

According to the present invention, the term “reducing body fat, promoting weight loss” used herein refers to measuring body weight, total fat mass, percentage of fat dry weight, subcutaneous fat and visceral fat in animal assay. Preferably, the plant extract composition can reduce above 7% lipid mass. More preferably, the plant extract composition of the present invention can reduce 15% to 70% lipid mass. Preferably, the plant extract composition of the present invention can reduce above 4% weight gain. More preferably, the plant extract composition of the present invention can reduce 10% to 60% weight gain.

According to the present invention, the term “increasing lipid metabolism” used herein refers to increase of CPT-1 activity to enhance carnitine binding with fatty acid, and thus facilitate fatty acid transport into mitochondria for oxidation lipid metabolism. According to the present invention, increasing lipid metabolism is determined by analyzing CPT-1 activity in animal assay; the plant extract composition of the present invention can increase CPT-1 activity from 3 folds to 4 folds. Besides, leptin resistance and lipogenesis induced by obesity can be improved by reducing obese genes expression, wherein the obese genes include, but are not limited to, leptin gene and resistin gene. According to the present invention, reducing obese genes expression is determined by analyzing the expression of leptin or resistin gene in animal assay. The plant extract composition in accordance with the present invention can reduce leptin gene expression from 25% to 50%, and reduce resistin gene expression from 15% to 80%.

In one embodiment of the present invention, the present invention also provides a method of a plant extract composition for inhibiting adipocytes, wherein adipocytes include, but are not limited to, preadipocytes, differentiating adipocytes, and mature adipocytes, wherein the therapeutically effective amount of the plant extract composition is from 10 mg/kg BW to 160 mg/kg BW.

Preferably, the therapeutically effective amount of the plant extract composition is from 26 mg/kg BW to 120 mg/kg BW.

According to the present invention, the term “effective amount” used herein refers to the announcement “estimating the maximum safe starting dose in initial clinical trials for therapeutics in adult healthy volunteers” of the US FDA and animal assay of the present invention.

In one embodiment of the present invention, the present invention also provides a method of the plant extract composition for reducing above 4% body weight gain and reducing above 7% fat mass, wherein the therapeutically effective amount of the plant extract composition is from 10 mg/kg BW to 160 mg/kg BW.

Preferably, the therapeutically effective amount of the plant extract composition is from 26 mg/kg BW to 120 mg/kg BW.

More preferably, the plant extract composition of the present invention can reduce 10% to 60% weight gain.

More preferably, the plant extract composition of the present invention can reduce 15% to 70% lipid mass.

In one embodiment of the present invention, the present invention also provides a method of the plant extract composition for increasing lipid metabolism, wherein the plant extract composition can activate carnitine palmitoyl transferase-1 (CPT-1), reduce the expression of leptin gene and resistin gene, wherein the therapeutically effective amount of the plant extract composition is from 10 mg/kg BW to 160 mg/kg BW.

Preferably, the therapeutically effective amount of the plant extract composition is from 26 mg/kg BW to 120 mg/kg BW.

Preferably, the plant extract composition can activate carnitine palmitoyl transferase-1 from 3 folds to 4 folds.

More preferably, the plant extract composition can reduce the expression of leptin gene from 25% to 50%. More preferably, the plant extract composition can reduce the expression of resistin gene from 15% to 80%.

In one embodiment of the present invention, the present invention also provides a pharmaceutical composition for inhibiting adipocytes, reducing body fat, promoting weight loss and increasing lipid metabolism, wherein the pharmaceutical composition comprises a therapeutically effective amount of the plant extract composition and a pharmaceutically acceptable carrier.

Preferably, the administration of the pharmaceutical composition of the present invention includes oral, parenteral, inhaled, anal suppositories, vaginal suppositories, transderma absorption or topical. The pharmaceutical composition comprises non-toxic pharmaceutically acceptable carriers, adjuvants and vehicles. Topical administration includes transdermal absorption, e.g., via skin patches or iontophoresis device. Parenteral administration includes subcutaneous injections, intravenous, intramuscular, intrasternal injections, or other injections.

In one embodiment, the pharmaceutical composition of the present invention is an oral solid formulation, including excipients, adhesive, disintegrants, lubricants, colorants or flavoring agents. The mixture may be formed by the conventional techniques to form tablets, granules, powders, capsules and the like. Additives may include lactose, sodium chloride, glucose, starch, calcium carbonate, kaolin, microcrystalline cellulose or silicic acid. Adhesive may include water, ethanol, propanol, sucrose solution, glucose solution, starch solution, gelatin solution, or phosphoric acid carbon. Disintegrants may include dry starch, sodium alginate, agar, sodium bicarbonate, calcium carbonate, sodium lauiyl sulfate, tallow, stearic acid monoglyceride, and lactose. Lubricants include talc, stearic acid salts, borax, and polyethylene glycol.

In another embodiment, the pharmaceutical composition of the present invention is an oral liquid formulation, wherein the pharmaceutical composition of the present invention includes flavoring agents, buffer, stabilizer, and the like. Buffer includes sodium citrate. Stabilizer includes tragacanth, arabic gum, or gelatin.

The content of the active ingredient of the present invention may be administered in accordance with different individuals, and the active ingredient can be combined with other carriers to form a single dose. The dose can be appropriately adjusted according to different parameters. Said parameters include, but are not limited to, the species of the individual, the size of the individual, and the severity of the disease. The active ingredient of the present invention may be administered once, repeatedly or continuously administrated within 24 hours. When administration is by continuous injection, a conventional and suitable means can be chosen, including, but not limited to, intravenous drip, intravenous pumps, implantable pump injection, or topical administration.

The pharmaceutical composition of the present invention can be administered alone or in combination with other drugs to lose weight.

The plant extract composition of the present invention can inhibit preadipocytes, differentiating adipocytes, and mature adipocytes, and increase the activity of carnitine acyl transferase-1, reduce lipid mass and promote weight loss. In addition, the composition of the present invention is able to significantly increase the activity of carnitine acyl transferase-1, inhibit lipogenesis, promote weight loss and lipolysis, and reduce the existing fat. Therefore, the composition in accordance with the present invention is able to reduce above 4% weight gain and reduce above 7% lipid mass. Because the plant extract composition of the present invention is considered highly safe, it causes no damage and has no side effects, such that the plant extract composition of the present invention can be used as a supplementary food or health food.

### BRIEF DESCRIPTION OF THE DRAWINGS

FIG. 1 shows the effect of the control group, green tea extract, resveratrol, green coffee bean extract and the formulations LB001, LB004, LB005, LB006, LB007, LB008, and LB008A of the present invention on the preadipocytes of the first embodiment of the present invention.

FIG. 2 shows the effect of the control group, green tea extract, resveratrol, green coffee bean extract and the formulations LB001, LB004, LB005, LB006, LB007, LB008, and LB008A of the present invention on the differentiating adipocytes of the second embodiment of the present invention.

FIG. 3 shows the effect of the control group, green tea extract, resveratrol, green coffee bean extract and the formulations LB001, LB004, LB005, LB006, LB007, LB008, and LB008A of the present invention on the mature adipocytes of the third embodiment of the present invention.

FIG. 4 shows the effect of the normal group, the obese group, low, medium, and high doses of the formulation LB004 of the present invention for the activity of rat CPT-1 of the fourth embodiment of the present invention.

FIG. 5 shows the effect of the normal group, the obese group, low, medium, and high doses of the formulation LB004 of the present invention on the expression of rat leptin gene of the fifth embodiment of the present invention.

FIG. 6 shows the effect of the normal group, the obese group, low, medium, and high doses of the formulation LB004 of the present invention on the expression of rat resistin gene of the fifth embodiment of the present invention.

FIG. 7 shows the effect of the normal group, the obese group, and the formulation LB004 of the present invention on mice body weight of the sixth embodiment of the present invention.

FIG. 8 shows the effect of the normal group, the obese group, the formulation LB004 of the present invention on mice visceral fat of the sixth embodiment of the present invention.

FIG. 9 shows the effect of the obese group, green tea extract, resveratrol, and the formulations LB001 and LB0008 of the present invention on mice weight gain of the seventh embodiment of the present invention.

FIG. 10 shows the effect of the obese group, green tea extract, resveratrol, and the formulations LB001 and LB0008 of the present invention on mice visceral fat, subcutaneous fat, and total fat mass of the seventh embodiment of the present invention.

### DETAILED DESCRIPTION OF THE PREFERRED EMBODIMENTS

Other objectives, advantages and novel features of the invention will become more apparent from the following detailed description when taken in conjunction with the accompanying drawings.

### Preparation Example 1

### Formulation of the Plant Extract Composition

TABLE 1
Formulation of the plant extract composition (wt %)

| | plant extract composition | | | | |
|---|---|---|---|---|---|
| | | | green coffee | mulberry | bitter |
| | resver- | green tea | bean | leaf | gourd |
| | atrol | extract | extract | extract | extract |
| LB001 | 29 | 71 | 0 | 0 | 0 |
| LB004 | 20.7 | 43.1 | 0 | 10.3 | 25.9 |
| LB005 | 17.2 | 17.2 | 0 | 34.5 | 46.6 |
| LB006 | 3.4 | 62.1 | 0 | 17.2 | 17.2 |
| LB007 | 20.7 | 10.3 | 0 | 8.6 | 60.3 |
| LB008 | 20 | 55 | 25 | 0 | 0 |
| LB008A | 25 | 50 | 25 | 0 | 0 |

### Example 1

### Preadipocytes Inhibition Assay

3T3-L1 preadipocytes (purchased from FIRDI, Taiwan) were seeded at a density of 1×10⁴ cells per well onto 96-well plate. Three repeated cell experiments were examined using 1% DMSO as control group and 50 ppm extract media (green tea extract, resveratrol, green coffee bean extract, the formulations LB001, LB004, LB005, LB006, LB007, LB008, and LB008A) with different concentrations were used. After incubation for 48 hours, the inhibitory effect on 3T3-L1 preadipocytes was analyzed by MTT assay.

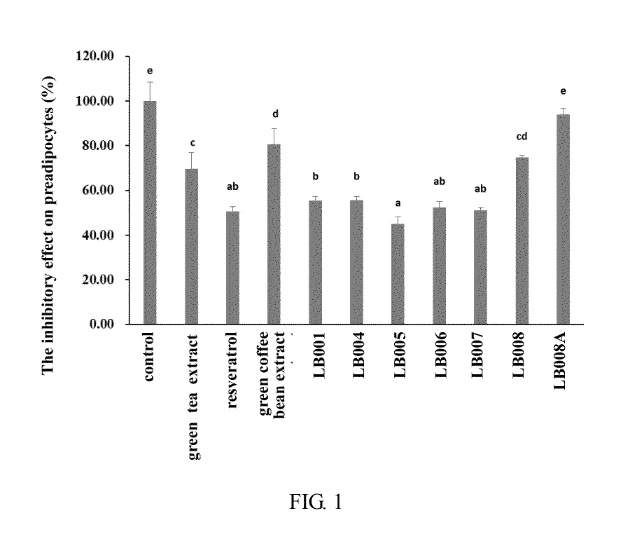

As shown in FIG. 1, the inhibitory effect on preadipocytes by the formulation LB001, LB004, LB005, LB006, and LB007 of the present invention was greater than that of the green tea extract or the green coffee bean extract, wherein the best inhibitory effect on 3T3-L1 preadipocytes was the formulation LB005. All data are presented as Mean±SD. The letters a, b, c, d, and e represent the results of the statistics, and the identical letter represents no statistical difference among the groups (p>0.05).

### Example 2

### Differentiating Adipocytes Inhibition Assay

3T3-L1 preadipocytes cells were seeded at a density of 3×10⁴ cells/cm² in 12-well plate. After the seeding for about four days, medium was replaced and contained 5 μg/ml insulin (differentiation agent), 1 μM dexamethasone, 0.5 mM 3-isobutyl-1-methylxanthine and 50 ppm extract (green tea extract, resveratrol, the formulations LB001, LB004, LB005, LB006, LB007, LB008, and LB008A, respectively. After incubation for another 48 hours, the inhibitory effect on differentiating adipocytes 3T3-L1 was analyzed by MTT assay.

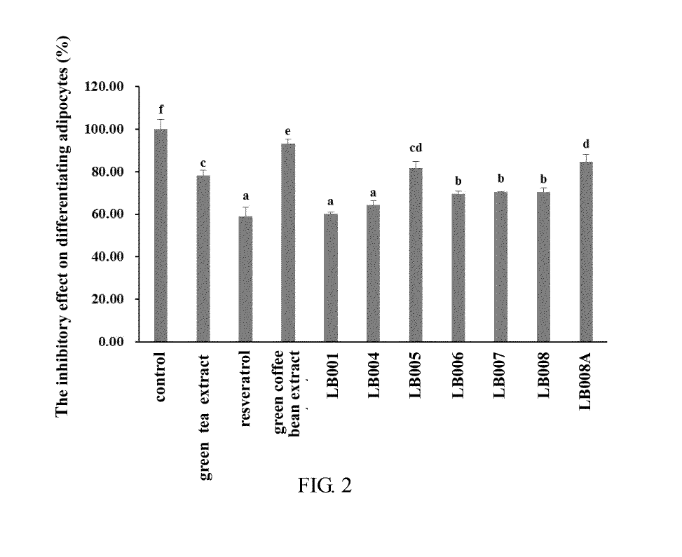

As shown in FIG. 2, the inhibitory effect on differentiating adipocytes by the formulations LB001, LB004, LB006, LB007 and LB008 of the present invention were all greater than that by the green tea extract and the green coffee bean extract, wherein the best inhibitory groups on differentiating adipocytes were formulations LB001 and LB004. All data are presented as Mean±SD. The letters a, b, c, d, e, and f represent the results of the statistics, and the identical letter represents no statistical difference among the groups (p>0.05).

### Example 3

### Mature Adipocytes Inhibition Assay

3T3-L1 cells were seeded at a density of 3×10⁴ cells/cm² in 12-well plate. After the seeding for about four days, medium was changed and contained 5 μg/ml insulin, 1 μM dexamethasone, and 0.5 mM 3-isobutyl-1-methylxanthine. After incubation for another two days, medium was renewed and contained 5 μg/ml insulin for additional four days maturity incubation. Medium containing 50 ppm extract (green tea extract, resveratrol, the formulations LB001, LB004, LB006, LB007, LB008 and LB008A) was respectively added to the 12-well plate. After incubation for 48 hours, the inhibitory effect on mature adipocytes 3T3-L1 was analyzed by MTT assay.

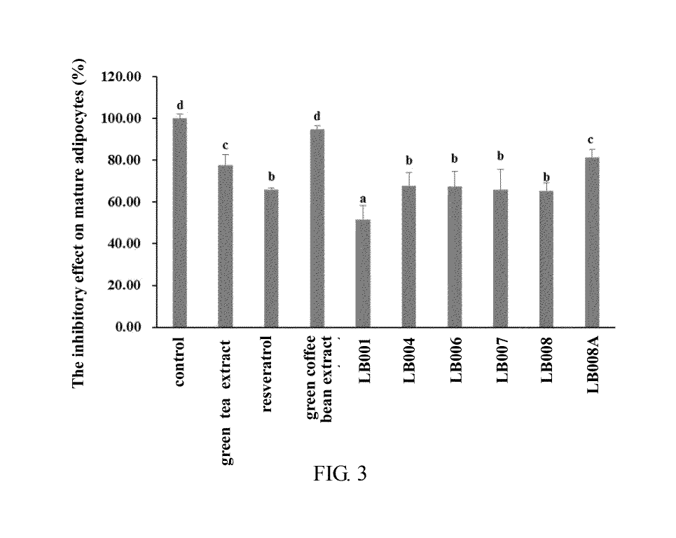

As shown in FIG. 3, the inhibitory effect on mature adipocytes by green tea extract or green coffee bean extract was minor while the inhibitory effect by the formulations LB001, LB004, LB006, LB007 and LB008 of the present invention was more relevant than that of the green tea extract and the green coffee bean extract, wherein the best inhibitory group on mature adipocytes was the formulation LB001. All data are presented as Mean±SD. The letters a, b, c, d, e, and f represent the results of the statistics and the identical letter represents no statistical difference among the groups (p>0.05).

### Example 4

### Animal Assay (I)

Sprague-Dawley (SD) male rats were used in this example. There were six groups for test, respectively as a normal group, an obese group, a positive control group (Orlistat®, 18.6 mg/kg BW), a low-dose experimental group (the formulation LB004: 223.2 mg/kg BW), a medium-dose experimental group (the formulation LB004: 446.4 mg/kg BW), and a high-dose experimental group (the formulation LB004: 892.8 mg/kg BW). Ten male rats were used in each group. During the 8-week experiment period, high fat diets were fed ad libitum for all groups except positive control group (Orlistat®). Three formulation LB004 doses of the present invention were orally given simultaneously during the experiment and body weight loss and lipid mass reduction were evaluated. Bodyweight, food and water intake were recorded weekly. In the end of the test, total amount of fat and the percentage of fat dry weight were determined.

In the end of the experiment, the rats were sacrificed. Epididymis fat, perinephric fat, and mesenteric fat were weighted and the total amount of internal fat was calculated. In addition, the fat of groin and the extraperitoneal fat were weighted to calculate the amount of subcutaneous fat.

In addition, the wet weights of rat bodies were weighed. Then the rat bodies were dried in an oven at 105° C. to constant dry weight for measuring dry weights of the rat bodies. The rat bodies were pulverized by uniform homogenizer. The percentage of dry weight and the amount of crude fat of whole animals is calculated by diethyl ether extraction at 2 to 3 different sampling points.

TABLE 2
Total weight gain, total fat mass, and
the fat percentage of dry bodyweight

| | total weight | total fat | fat (dry |
|---|---|---|---|
| Group | gain (g) | mass (g) | weight)(%) |
| normal group | 201.7 ± 22.6^{a} | 28.9 ± 2.8^{a} | 29.3 ± 2.5^{a} |
| obese group | 272.9 ± 19.8^{b} | 50.1 ± 8.4^{b} | 38.3 ± 2.9^{b} |
| positive control group | 240.1 ± 24.4^{c} | 38.1 ± 8.8^{c} | 31.6 ± 6.0^{a} |
| (Orlistat ®, | | | |
| 18.6 mg/kg B.W.) | | | |
| low-dose experimental | 237.8 ± 32.3^{c} | 37.5 ± 7.1^{c} | 30.8 ± 4.6^{a} |
| group (formulation | | | |
| LB004: 223.2 | | | |
| mg/kg B.W.) | | | |
| medium-dose experi- | 234.5 ± 20.9^{c} | 37.1 ± 11.0^{c} | 30.8 ± 4.1^{a} |
| mental group (formula- | | | |
| tion LB004: 446.4 | | | |
| mg/kg B.W.) | | | |
| high-dose experimental | 225.2 ± 21.1^{c} | 36.7 ± 4.9^{c} | 31.1 ± 3.7^{a} |
| group (formulation | | | |
| LB004: 892.8 | | | |
| mg/kg BW) | | | |

As shown in Table 2, the weight gain and body fat of the obese group (the sum of visceral fat and subcutaneous fat) was significantly higher than those of the normal group (p<0.05). Compared with that of the obese group, the body weight of the positive control (Orlistat®) and three formulation LB004 experimental groups in accordance with the present invention was significantly decreased (p<0.05). Compared with that of the obese group, the body weight decline of the positive control group, low-dose, medium-dose, and high-dose body weight decline in the experimental group were decreased, respectively by 12.0%, 12.8%, 14.1% and 17.5%. Compared with the obese group, the body fat of the positive control group (Orlistat®) and three experimental groups of formulation LB004 in accordance with the present invention were significantly decreased (p<0.05). Compared with the obese group, the body fat of the positive control group, low-dose, medium-dose, and high-dose body weight decline in the experimental group were decreased, respectively by 23.9%, 25.1%, 25.9%, and 26.7%. As the test results show, the efficacy of decreasing body fat of the formulation LB004 in accordance with the present invention was greater than Orlistat® in trend. In addition, the percentage of fat dry weight of the obese group was significantly higher than the normal group (p<0.05). Compared with the obese group, the percentage of fat dry weight of the positive control group (Orlistat®) and three experimental groups of formulation LB004 in accordance with the present invention showed a significant decrease (p<0.05), respectively by 17.4%, 19.6%, 19.7%, and 19.0%. The experimental results showed that the efficacy of the formulations of the present invention was superior to Orlistat®.

### Example 5

### Animal Assay (II)

The method is the same as in Example 4. There were five groups, respectively the normal group, the obese group, the low-dose experimental group (the formulation LB004: 223.2 mg/kg BW), the medium-dose experimental group (the formulation LB004: 446.4 mg/kg BW), and the high-dose experimental group (the formulation LB004: 892.8 mg/kg BW). The activity of CPT-1 in liver, the gene expression of lectin and resistin were compared in the above groups.

5.1 CPT-1 Activity Assay (in Liver):

The liver tissues were homogenized by homogenization buffer I (containing 250 mM sucrose, 1 mM EDTA, and 10 mM Tris-HCl, pH 7.4), and then centrifuged for 10 minutes. After the supernatant was removed, the liver tissues were dissolved in homogenization buffer II (containing 70 mM sucrose, 220 mM mannitol, 1 mM EDTA, and 2 mMHEPES, pH 7.4), and then mitochondrial proteins were extracted.

By means of CPT in rat liver mitochondria, carnitine combining with palmitoyl-CoA can be reduced to form CoA-SH, the activity of CPT-1 can be detected from yellow product 5-thio-2-nitrobenzoate (TNB) formed from 5,5′-dithio-bis(2-nitrobenzoic acid) (DTNB) combining with sulfhydryl (—C—SH or R—SH) group at 412 nm, and referred to as CoA-SH (nmole)/min/mg protein. The activity of CPT-2 can be detected in the same way, and CPT-1 inhibitor was added in the last step of the experiment.

The CPT-1 activity was obtained from total CPT activity excluding CPT-2 activity, and CPT concentrations can be converted by Beer's law as follows:

A=εbc, wherein A is the absorbance at a given wavelength of light (M⁻¹cm⁻¹), ε is the molar absorption, b is the length of light path through the sample (cm), and c is the concentration.

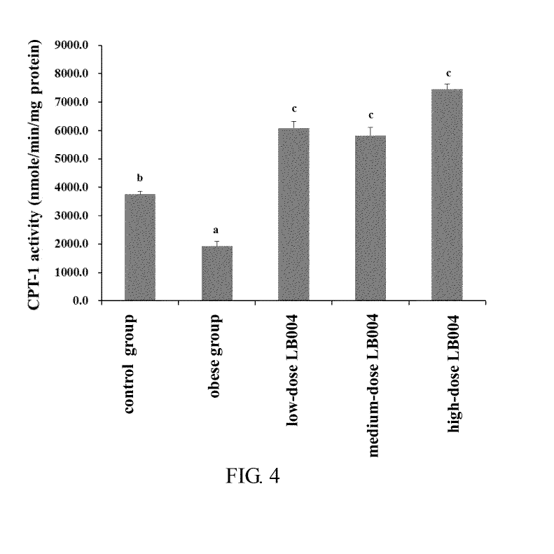

As shown in FIG. 4, compared with the normal group, the activity of CPT-1 was significantly decreased in the obese group. Compared with the obese group, the activities of CPT-1 of the formulations LB004 with low-dose, medium-dose, and high dose of the experimental groups of the present invention were significantly increased, so that the formulations of the present invention were effective to increase the activity of CPT-1, and to promote lipid metabolism. Compared with the obese groups, the formulation LB004 of the present invention can increase CPT-1 activity by 3 folds to 4 folds.

5.2 Rat Leptin Gene Expression Assay:

There were five groups for test, respectively the normal group, the obese group, the low-dose experimental group (the formulation LB004: 223.2 mg/kg BW), the medium-dose experimental group (the formulation LB004: 446.4 mg/kg BW), and the high-dose experimental group (the formulation LB004: 892.8 mg/kg BW), and the expressions of leptin gene were compared in the above groups.

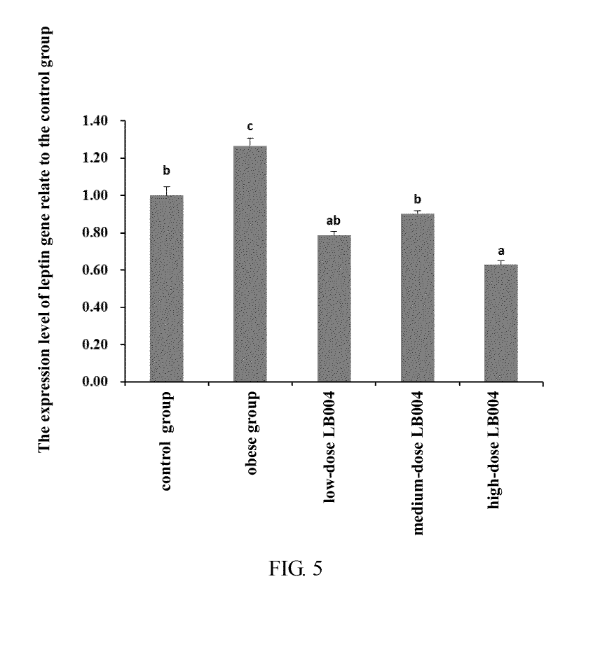

As shown in FIG. 5, compared with the normal group, the expression of leptin gene of the obese group was significantly increased. Compared with the obese group, the expression of leptin gene of the formulations LB004 with low-dose, medium-dose, and high dose of the experimental groups of the present invention were significantly decreased. Compared with the obese group, the formulation LB004 in accordance with the present invention can reduce leptin gene expression from about 25% to about 50%. The results showed that the formulations of the present invention were effective to increase the expression of leptin gene, and resolve issues of leptin resistance caused by obesity.

5.3 Rat Resistin Gene Expression Assay:

There were five groups for test, respectively the normal group, the obese group, the low-dose experimental group (the formulation LB004: 223.2 mg/kg BW), the medium-dose experimental group (the formulation LB004: 446.4 mg/kg BW), and the high-dose experimental group (the formulation LB004: 892.8 mg/kg BW), and the expressions of resistin gene were compared in the above groups.

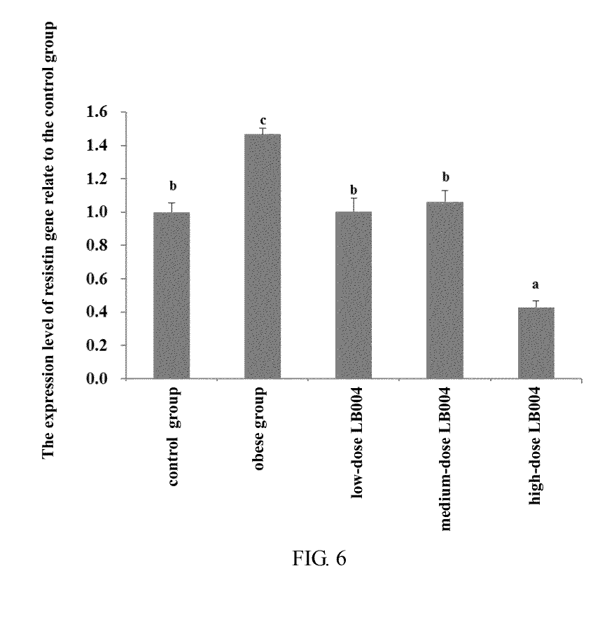

Resistin is a cytokine secreted by adipocytes, would cause insulin resistance, and is considered a risk factor of obesity. As shown in FIG. 6, compared with the normal group, the expression of resistin gene of the obese group was significantly increased. Compared with the obese group, the expressions of resistin gene of the formulations LB004 with low-dose, medium-dose, and high dose of the experimental groups of the present invention were significantly decreased. Compared with the obese group, the formulation LB004 in accordance with the present invention can reduce resistin gene expression from about 15% to about 80%. The results showed that the formulations of the present invention were effective to increase the expression of resistin gene and to inhibit lipogenesis.

### Example 6

### Animal Assay (III)

B6 strain male mice were used in this example. There were three groups for test, respectively as the control group, the obese group, and an experimental group (the formulation LB004: 422.8 mg/kg BW). Eight male animals were used in each group. During the test, high fat diets were fed for the obese group and the experimental group to induce obesity during sixteen weeks. In the experimental duration, weight and the average daily intake were recorded. In the end of the experiment, the visceral fat of the mice were determined, and then the mice were sacrificed to weight epididymis fat, perinephric fat, and mesenteric fat and calculate the total amount of visceral fat.

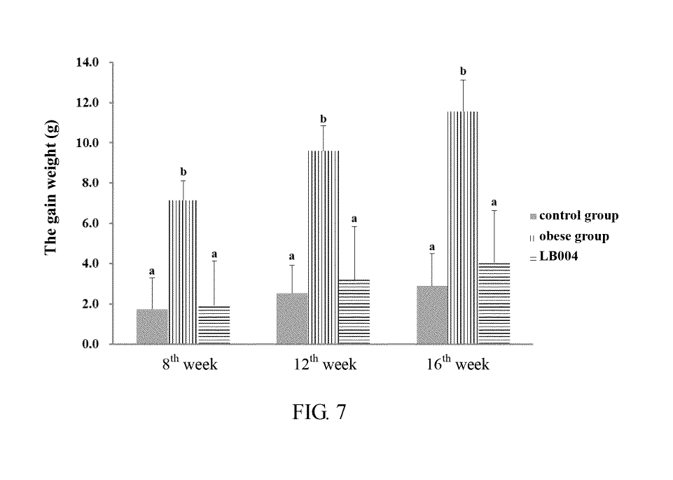

As shown in FIG. 7, the weight gain of the obese group was significantly higher than the normal group at the 8^{th}, 12^{th}, and 16^{th} weeks (p<0.05). Compared with the obese group, the weight of the experimental group was significantly decreased (p<0.05). The results showed that although mice were administered with high-fat diet during 16 weeks, the total weight gain had no significant difference between the normal group and the experimental group (administered with the formulation LB004), which means that the formulation LB004 in accordance with the present invention can inhibit weight gaining effectively.

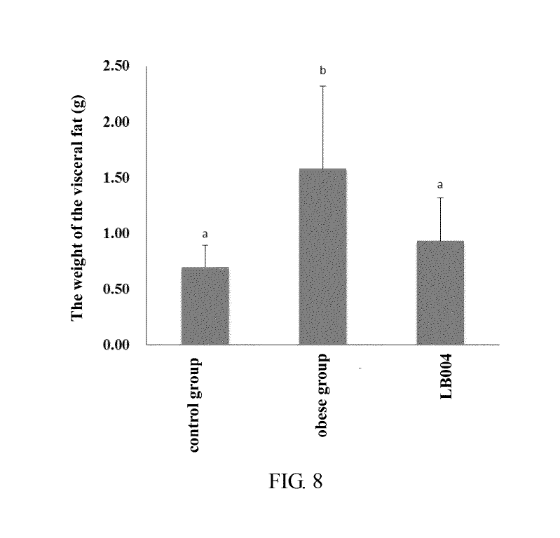

As shown in FIG. 8, the weight of the visceral fat of the obese group was significantly higher than the control group after sixteen weeks (p<0.05), whereas the weight of the visceral fat of the experimental group was significantly decreased compared with the obese group (p<0.05). The weight of visceral fat of the experimental group had no significant differences from the control group. The results showed that the formulation LB004 in accordance with the present invention can inhibit growth of visceral fat effectively.

### Example 7

### Animal Assay (IV)

B6 strain male mice were used in this example. There were five groups for test, respectively as the obese group, the resveratrol (61.5 mg/kg BW), the green tea extract (123 mg/kg BW), and experimental groups such as the formulation LB001 (338.25 mg/kg BW) and the formulation LB0008 (326 mg/kg BW). Five male animals were used in each group. During the test, high fat diets and experimental substrates were fed for all groups to induce obesity during eight weeks. In the experimental duration, weight and the average daily intake were recorded. In the end of the experiment, the mice were sacrificed to weight epididymis fat, perinephric fat, and mesenteric fat and calculate the total amount of visceral fat; groin fat and extra-abdominal fat were weighed to calculate the amount of subcutaneous fat.

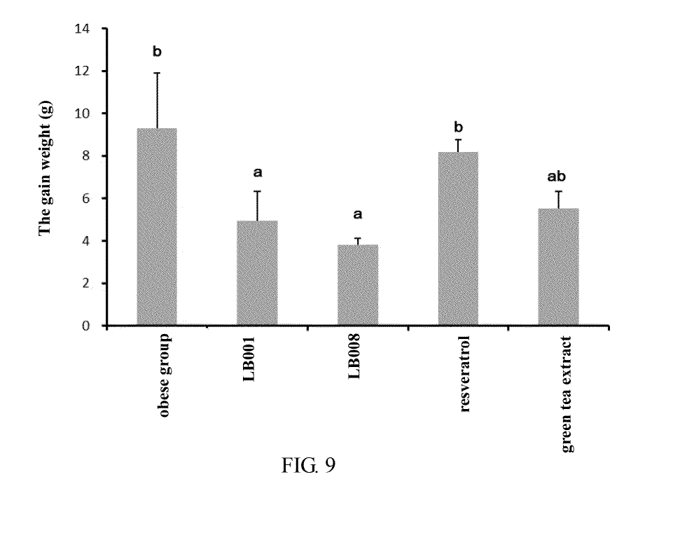

As shown in FIG. 9, the inhibitory effect on weight gain by the resveratrol alone was poor and had no statistical difference from the obese group. Compared with the obese group, the weight gains of the formulations LB0001 and LB0008 in accordance with the present invention were decreased significantly, and the inhibitory effect of weight gain of the formulation LB0001 and LB008 were 47.2% and 59.2% respectively. Even the inhibitory effect on body fat and weight gain of the formulations LB001 and LB008 were much better than green tea extract and resveratrol. All data are Mean±SD (n=5). The letters a, b, and c represent the results of the statistics, the identical letter represents no statistical difference among the groups (p>0.05).

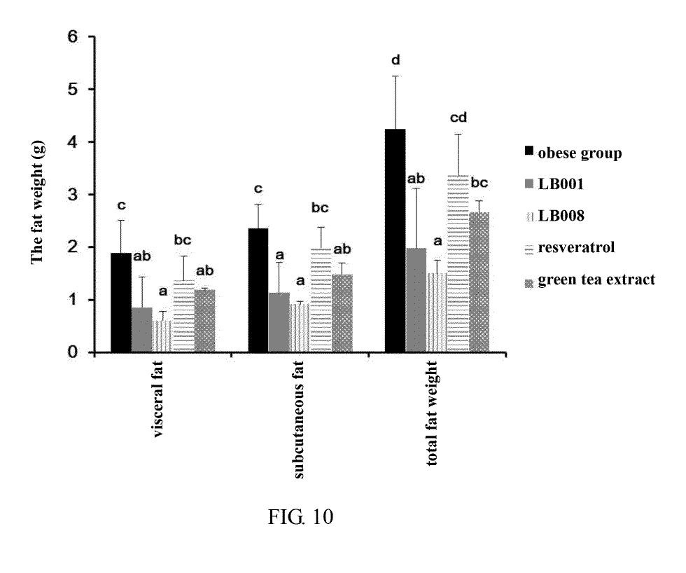

As shown in FIG. 10, compared with the obese group, visceral fat, subcutaneous fat, and total fat mass (the sum of subcutaneous fat and visceral fat) of the resveratrol had no statistical difference. Compared with the obese group, visceral fat, subcutaneous fat, and total fat mass of the formulations LB001 and LB0008 in accordance with the present invention were significantly decreased (p<0.05), wherein the formulation LB001 can reduce subcutaneous fat by 54.8%, visceral fat by 52.3%, and total fat mass by 53.4%; wherein the formulation LB008 can reduce subcutaneous fat by 68.1%, visceral fat by 61.6%, and total fat mass by 64.7%. Compared with the resveratrol, visceral fat, subcutaneous fat, and total fat mass of the formulations LB001 and LB0008 in accordance with the present invention were significantly decreased (p<0.05). The results showed that the formulations LB001 and LB008 can effectively inhibit lipogenesis, and that effect was significantly better than resveratrol alone.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A plant extract composition for inhibiting adipocytes, reducing body fat, promoting weight loss and increasing lipid metabolism, wherein the plant extract composition comprises 1.5 wt % to 40 wt % resveratrol and 10 wt % to 85 wt % green tea extract.

2. The composition as claimed in claim 1, wherein the plant extract composition further comprises 10 wt % to 30 wt % resveratrol.

3. The composition as claimed in claim 1, wherein the plant extract composition further comprises 40 wt % to 80 wt % green tea extract.

4. The composition as claimed in claim 1, wherein the plant extract composition further comprises an extract selected from the group consisting of green coffee bean extract, mulberry leaf extract and bitter gourd extract, wherein green coffee bean extract is from 0 wt % to 40 wt %, mulberry leaf extract is from 0 wt % to 40 wt %, and bitter gourd extract is from 0 wt % to 65 wt %.

5. The composition as claimed in claim 1, wherein the plant extract composition comprises 20 wt % to 40 wt % green coffee bean extract.

6. The composition as claimed in claim 1, wherein the plant extract composition comprises 10 wt % to 40 wt % mulberry leaf extract.

7. The composition as claimed in claim 1, wherein the plant extract composition comprises 17 wt % to 50 wt % bitter gourd extract.

8. A method of the plant extract composition as claimed in claim 1 for inhibiting adipocytes, wherein the plant extract composition can inhibit preadipocytes, differentiating adipocytes, and mature adipocytes, wherein a therapeutically effective amount of the plant extract composition is from 10 mg/kg BW to 160 mg/kg BW.

9. The method as claimed in claim 8, wherein, the therapeutically effective amount of the plant extract composition is from 26 mg/kg BW to 120 mg/kg BW.

10. A method of the plant extract composition as claimed in claim 1 for reducing body fat and promoting weight loss, wherein a therapeutically effective amount of the plant extract composition is from 10 mg/kg BW to 160 mg/kg BW.

11. The method as claimed in claim 10, wherein, the therapeutically effective amount of the plant extract composition is from 26 mg/kg BW to 120 mg/kg BW.

12. A method of the plant extract composition as claimed in claim 1 for increasing lipid metabolism, wherein the plant extract composition can increase CPT-1 activity, reduce expression of leptin gene or resistin gene, wherein a therapeutically effective amount of the plant extract composition is from 10 mg/kg BW to 160 mg/kg BW.

13. The method as claimed in claim 14, wherein, the therapeutically effective amount of the plant extract composition is from 26 mg/kg BW to 120 mg/kg BW.

14. A pharmaceutical composition for inhibiting adipocytes, reducing body fat, promoting weight loss and increasing lipid metabolism, wherein the pharmaceutical composition comprises a therapeutically effective amount of the plant extract composition as claimed in claim 1 and a pharmaceutically acceptable carrier.
